(19) United States
(12) Patent Application Publication
(10) Pub. No.: US 2012/0046610 A1
(43) Pub. Date: Feb. 23, 2012

(54) METHODS AND DEVICES FOR REDUCING BUBBLE FORMATIONS IN FLUID DELIVERY DEVICES

(75) Inventor: DARRELL RANKIN, MILPITAS, CA (US)

(73) Assignee: BOSTON SCIENTIFIC SCIMED, INC., MAPLE GROVE, MN (US)

(21) Appl. No.: 13/210,067

(22) Filed: Aug. 15, 2011

Related U.S. Application Data
(60) Provisional application No. 61/374,533, filed on Aug. 17, 2010.

(51) Int. Cl. A61M 3/02; A61B 18/14

(52) U.S. Cl. 604/122; 606/41

(57) ABSTRACT

Methods and devices for filtering a fluid flowing through a medical device are disclosed. In one example, a medical device may include a catheter shaft including a proximal region having a coupling for coupling to a fluid source and a distal region including one or more irrigation apertures for expelling a fluid from the catheter. A fluid path can be defined by the catheter shaft between the coupling and the one or more irrigation apertures. A porous member can be positioned at a location in the fluid path such that the fluid being expelled from the catheter via the one or more irrigation apertures may flow through the porous member to filter, reduce, and/or break-up bubble formations in the fluid.

### CROSS-REFERENCE TO RELATED APPLICATIONS

This application claims the benefit of U.S. Provisional Application Ser. No. 61/374,533, filed Aug. 17, 2010, the entire disclosure of which is incorporated herein by reference.

### FIELD

The present disclosure relates generally to medical devices and, more particularly, to methods and devices for filtering, reducing, and/or breaking up bubble formations in a fluid delivery medical device.

### BACKGROUND

A variety of minimally invasive electrophysiological procedures employing catheters and other apparatus have been developed to treat conditions within the body by ablating soft tissue (i.e. tissue other than blood, bone and connective tissue). With respect to the heart, minimally invasive electrophysiological procedures have been developed to treat atrial fibrillation, atrial flutter and ventricular tachycardia by forming therapeutic lesions in heart tissue. The formation of lesions by the coagulation of soft tissue (also referred to as “ablation”) during minimally invasive surgical procedures can provide the same therapeutic benefits provided by certain invasive, open heart surgical procedures. Atrial fibrillation has, for example, been treated by the formation of one or more long, thin lesions in heart tissue. The treatment of atrial flutter and ventricular tachycardia, on the other hand, requires the formation of relatively large lesions in heart tissue.

For some of these procedures, an ablation catheter is typically advanced into the heart via the patient's vessels. When electrodes of the ablation catheter are placed in the desired position within the heart chamber, radio frequency (RF) energy can be supplied to the electrodes thereby forming lesions into the soft tissue. However, the RF energy may cause the ablation catheter to overheat causing hot spots, coagulation, and/or other problems. In some procedures, a cooling fluid can be delivered to the distal tip of the ablation catheter and/or into the vessel or heart to help reduce problems associated with overheating. However, fluid delivery may cause its own problems, such as, for example, the formation of air embolisms. Therefore, there is a need for new and improved fluid delivery devices.

### BRIEF SUMMARY

The present disclosure relates generally to medical devices and, more particularly, to methods and devices for filtering, reducing, and/or breaking up bubble formations in a fluid delivery medical device. In one illustrative embodiment, a medical device may include an elongated shaft including a proximal region and a distal region. The proximal region of the elongated shaft may be configured to be coupled to a fluid source for receiving a cooling fluid and the elongate shaft may also define at least one cooling lumen in fluid communication with the fluid source for supplying the cooling fluid to the distal region of the catheter shaft. An electrode tip may be positioned adjacent to the distal region of the elongated shaft. The electrode tip may include a wall defining a cooling chamber that is in fluid communication with the at least one cooling lumen. The wall may include one or more irrigation apertures for expelling the cooling fluid from the cooling chamber of the electrode tip. A porous member may be disposed in the cooling chamber or the at least one cooling lumen such that substantially all of the cooling fluid that is expelled through the one or more irrigation apertures flows through the porous member prior to being expelled. The porous member may include a plurality of pores sized and configured to filter, reduce, and/or break-up bubble formations in the cooling fluid such that bubble formations posing a risk of forming embolisms in a vessel or body cavity may not be expelled through the one or more irrigation apertures.

In another illustrative embodiment, a medical device may include a catheter including a proximal region and a distal region. The proximal region of the catheter may include a coupling configured to couple to a fluid source for receiving a fluid and the distal region of the catheter may include one or more irrigation apertures for expelling the fluid from the catheter. The catheter may also define a fluid path extending between the coupling and the one or more irrigation apertures. A porous member, which may include a plurality of micro-pores, may be positioned at a location in the fluid path and may be configured to substantially fill the cross-sectional area of the location in the fluid path such that the fluid being expelled from the catheter via the one or more irrigation apertures may flow through the plurality of micro-pores.

In another illustrative embodiment, a method of delivering a fluid to a treatment site with a fluid delivery device may include coupling a proximal end of the fluid delivery device to a fluid source, providing a fluid flow through a fluid path of the fluid delivery device, passing the fluid flow through a porous member positioned in the fluid path to filter the fluid flow for bubble formations, and expelling the filtered fluid flow from the fluid delivery device through one or more irrigation apertures. In some cases, the method may also include providing an electrical signal to an electrode positioned in the distal region of the fluid delivery device via one or more electrical conductors and ablating tissue adjacent to the distal region of the fluid delivery device.

The preceding summary is provided to facilitate an understanding of some of the innovative features unique to the present disclosure and is not intended to be a full description. A full appreciation of the disclosure can be gained by taking the entire specification, claims, drawings, and abstract as a whole.

### BRIEF DESCRIPTION OF THE DRAWINGS

The disclosure may be more completely understood in consideration of the following detailed description of various illustrative embodiments of the disclosure in connection with the accompanying drawings, in which:

FIG. 1 is a schematic diagram of an illustrative fluid delivery system;

FIG. 2 is a perspective view of an illustrative embodiment of an ablation catheter;

FIGS. 3 and 4 are partial cut-away views of the distal region of the illustrative ablation catheter of FIG. 2;

FIG. 5 is a cross-sectional view of the distal region of the illustrative ablation catheter of FIGS. 2-4;

FIG. 6 is a perspective view of an illustrative filter that may be used in the distal tip of the ablation catheter of FIGS. 2-5;

FIG. 7 is a cross-sectional view of another illustrative distal region that may be used with the ablation catheter of FIG. 2.

### DETAILED DESCRIPTION

The following description should be read with reference to the drawings wherein like reference numerals indicate like elements throughout the several views. The detailed description and drawings, which are not necessarily drawn to scale, show several embodiments which are meant to be illustrative and are not intended to limit the scope of the disclosure.

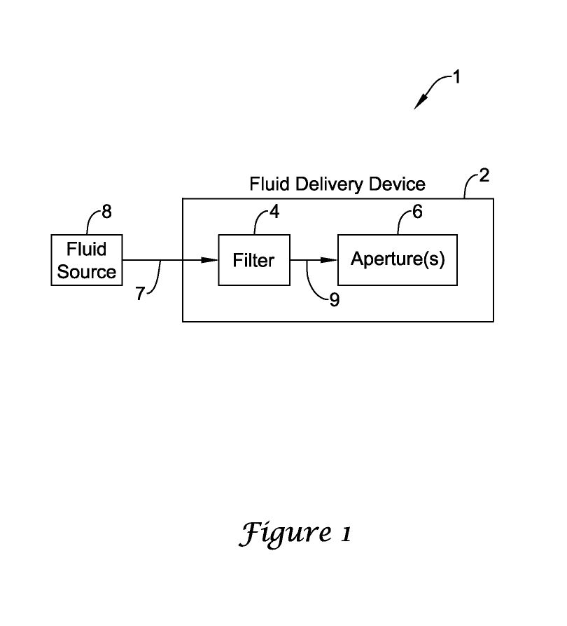

FIG. 1 is a schematic diagram of an illustrative embodiment of a fluid delivery system 1. In the illustrative embodiment, the fluid delivery system 1 may include a fluid delivery device 2 in fluid communication with a fluid source 8 for receiving a fluid 7. The illustrative fluid delivery device 2 may be a medical device that is configured to be advanced through a vessel to perform a minimally invasive electrophysiological or other medical procedure that emits fluid into the vessel during the procedure. An example fluid delivery device may be an ablation catheter, such as an open-irrigated ablation catheter. However, it is contemplated that the fluid delivery device 2 may include any other medical device that emits a fluid prior to, during, or after a medical procedure.

As illustrated in FIG. 1, the fluid delivery device 2 includes a filter 4 for filtering the fluid 7 received from the fluid source 8 to provide a filtered fluid 9. The filtered fluid 9 can then be expelled from the fluid delivery device 2 via one or more apertures 6. In the illustrative embodiment, the filter 4 can be configured to remove, filter, break-up, reduce, and/or eliminate the presence of gas formations, such as bubbles, in the fluid 7 supplied by the fluid source 8. Filtering fluid 7 for bubbles may help to reduce the formation of air embolisms in the vessel or other portion of the body during and/or after the medical procedure.

In the illustrative embodiment, the filter 4 may be positioned at any location between the fluid source 8 and the one or more apertures 6. For example, the filter 4 may be positioned at an interface between the fluid delivery device 2 and the fluid source 8, at a proximal end of the fluid delivery device 2, in a proximal region of the fluid delivery device 2, in a distal region of the fluid delivery device 2, at a distal end of the fluid delivery device, and/or at any other location between the fluid source 8 and the one or more apertures 6, as desired.

In the illustrative embodiment, filter 4 may include any material having a porosity that allows fluid to flow through, but that filters, breaks-up, reduces, and/or eliminates bubbles in the fluid. In some cases, filter 4 may include porous material having a plurality of pores. In some cases, the porous material may include two or more pores, three or more pores, four or more pores, five or more pores, six or more pores, seven or more pores, eight or more pores, nine or more pores, ten or more pores, twenty or more pores, or any other number of pores, as desired. In some instances, the plurality of pores may be oriented in a parallel configuration or, in other instances, in a non-parallel configuration. In some cases, the plurality or pores, or at least two pores, may be arranged in a parallel configuration. In some instances, the plurality of pores may be arranged in a generally uniform configuration or, in other instances, may be arranged in a generally non-uniform configuration. The plurality of pores may be sized to allow fluid flow therethrough while filtering the fluid for bubbles. In one example, the diameter of the plurality of pores may be on the order of micrometers. However, it is contemplated that any suitable diameter may be used that may be sufficient to filter the fluid for bubbles such that any remaining bubbles may not pose a significant risk of causing air embolisms in the vessel or other portion of the body.

Example materials that may be used for filter 4 may include, but are not limited to, a fabric, a membrane, a woven mesh, a non-woven fiber, a sintered material, a porous fiber such as a porous carbon fiber, and/or any other suitable porous material. The filter 4 may include, for example, a metal, a ceramic, a polymer, and/or other suitable material. Porous polymer materials may include, for example, thermoset polymers, thermoplastic polymers, elastomer materials, organic or synthetic materials, and any other suitable polymer material, as desired. However, the foregoing materials are merely illustrative and are not meant to be limiting in any manner. It is to be understood that any suitable porous material may be used for filter 4, as desired.

In addition, while only one filter 4 is shown in FIG. 1, it is contemplated that multiple filters 4 can be positioned in one or multiple locations of the fluid delivery device, as desired.

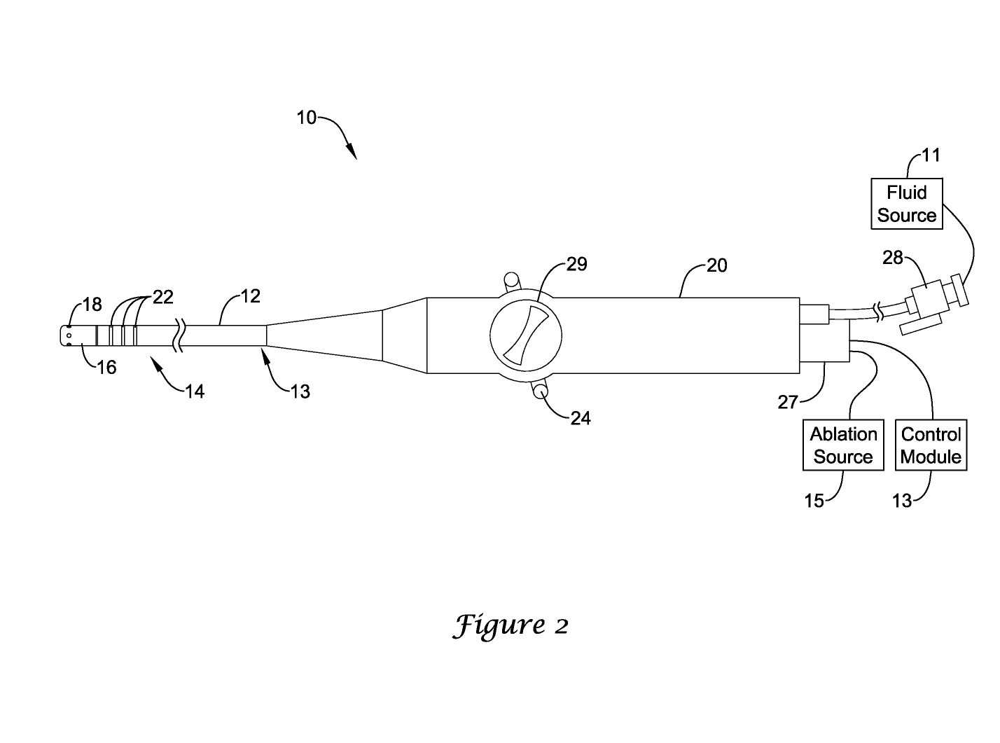

FIGS. 2-8 are illustrative embodiments of an ablation catheter including a filter in accordance with the present disclosure. However, ablation catheters are just one example and it is contemplated that the filter may be incorporated into other medical devices that emit a fluid. FIG. 2 is a perspective view of an illustrative ablation catheter 10. In some embodiments, the ablation catheter 10 can be an open-irrigated ablation catheter or, in other words, an ablation catheter that delivers fluid through one or more apertures in the tip of the catheter 10.

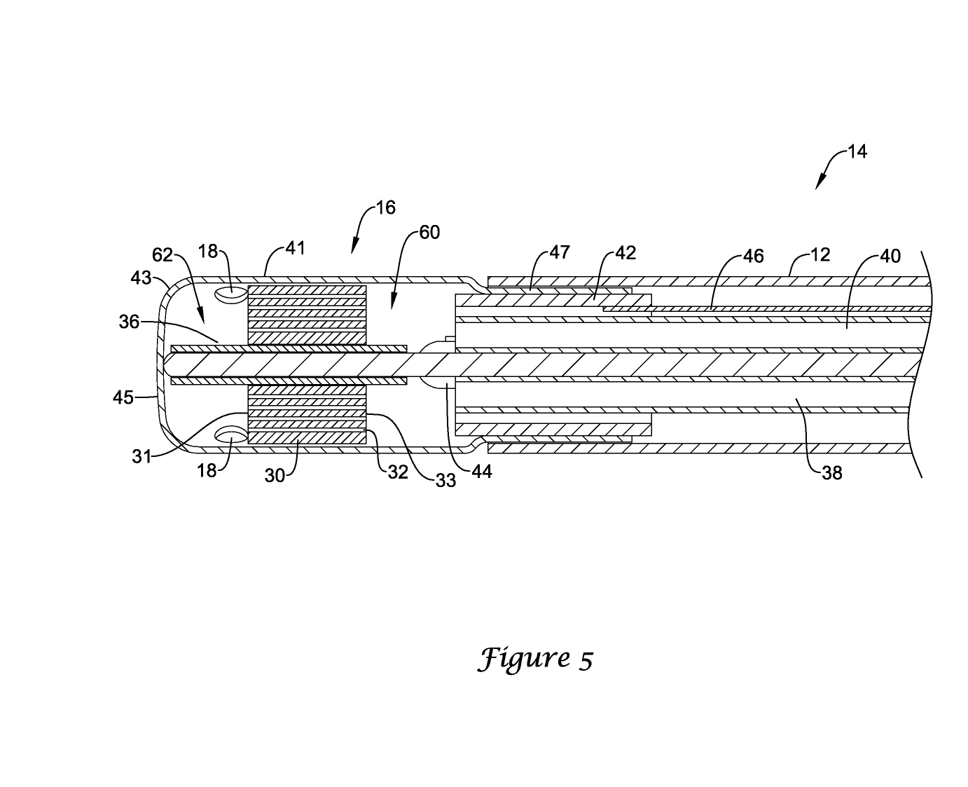

In the illustrative embodiment, ablation catheter 10 may include an elongated tubular member or shaft 12 including a proximal section 13 and a distal section 14. The elongated shaft 12 may be configured to include one or more fluid passageways for delivering a fluid, such as a cooling fluid, to a distal tip 16 and, in some cases, returning the cooling fluid from the distal tip 16. In some embodiments, the elongated shaft 12 may include one or more electrical conductors (e.g., wires) (shown as 46 in FIG. 5) for transmitting electrical signals to the distal section 14 of the ablation catheter 10 related to sensing and/or ablating of the tissue. In some embodiments, the elongated shaft 12 may include one or more articulation mechanisms, such as, for example, pull wires, for articulating at least a portion of the elongated shaft 12, but this is not required.

In some embodiments, the elongated shaft 12 may be formed from one or more sections of material to help achieve desired characteristics of the elongated shaft 12, such as, for example, pushability, torqueability, and/or flexibility. In the illustrative embodiment, the elongated shaft 12 may include a proximal section 13 including a first material and a distal section 14 including a second material. However, it is contemplated that elongated shaft 12 may include a single material along its length or may include additional sections of materials, as desired.

In the illustrative example, proximal section 13 of the elongated tubular member may include a material to impart flexibility and stiffness characteristics according to the desired application. In the illustrative embodiment, the proximal section 13 may include a material to impart stiffness, pushability, and/or torqueability in the catheter 10. For example, the proximal section 13 may include a rigid and resilient material. In such an embodiment, the proximal section 13 may be made from a metal, a metal alloy, a polymer, a metal-polymer composite, and the like, or any other suitable material. In one example, the proximal region may include a thermoplastic material. For example, the proximal section 13 may include metal-polymer composite such as, for example, polyether block amide (PEBA, for example available under the trade name PEBAX®) and a stainless steel braid composite or a polyethylene and a stainless steel braid composite. However, these materials are just examples and are not meant to be limiting in any manner. It is to be understood that the proximal section 13 may include any suitable material commonly used in medical devices, as desired.

In the illustrative embodiment, the distal section 14 of the elongated shaft 12 may be disposed distally of the proximal section 13 and bonded (e.g. adhesively, thermally, etc.) or otherwise connected to the proximal section 13. The distal region may include a material to impart flexibility and stiffness characteristics according to the desired application. For example, the distal section 14 may include a relatively softer and more flexible material than the proximal region 14. In such an embodiment, the distal section 14 may be made from a metal, a metal alloy, a polymer, a metal-polymer composite, and the like, or any other suitable material. In one example, the distal region may include an unbraided polyether block amide (PEBA, for example available under the trade name PEBAX®), polyethylene, or polyurethane. However, these are just examples and are not meant to be limiting in any manner. It is to be understood that the distal section 14 may include any suitable material commonly used in medical devices, as desired.

Additionally, the foregoing elongated shaft 12 is merely illustrative and is not meant to be limiting in any manner. It is to be understood that any suitable elongated member may be used in the catheter 10, as desired. For example, it is contemplated that elongated shaft 12 may include one or more guide coils, markers, and/or other features, as desired.

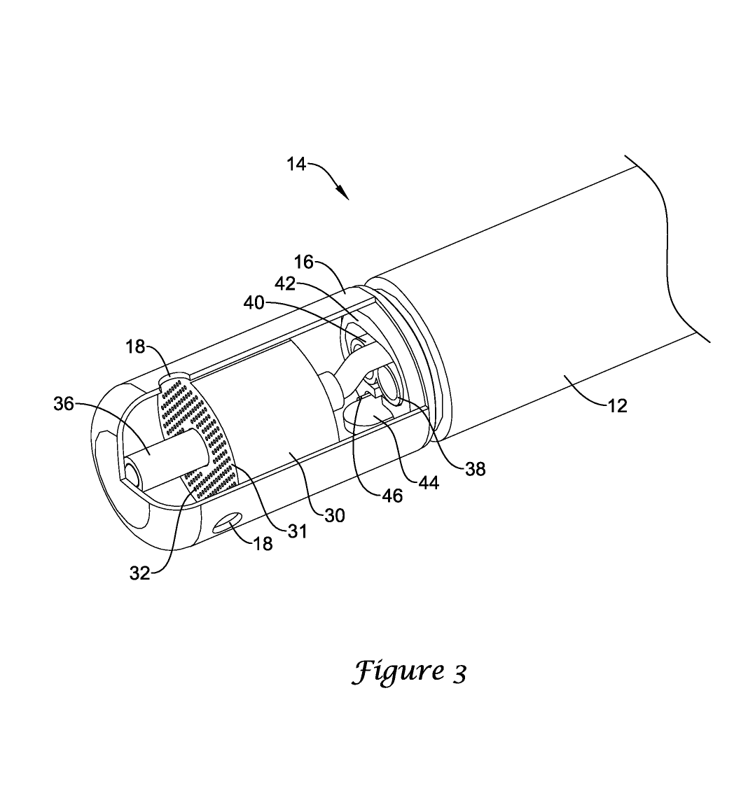

In the illustrative embodiment, a distal tip 16 and/or distal section 14 of the elongated shaft 12 may include one or more electrodes for delivering ablation energy, sensing physiological signals, and/or acting as a return electrode. As shown in FIG. 2, catheter 10 may include one or more ring electrodes 22 positioned around a portion of the distal section 14 of the catheter 10. For simplicity, ring electrodes 22 are not shown in FIGS. 3-5, but may still be provided as desired. Additionally, distal tip 16 may form an electrode tip of the ablation catheter 10 to, for example, deliver ablation energy. When provided, the electrodes 22, which may be used for electrical sensing or tissue ablation, can be connected to an electrical connector 27 on the handle 20 by one or more electrical conductors or wires extending through the elongated shaft 12. The electrodes 22 may include a conductive material, such as, for example, silver, platinum, gold, stainless steel, plated brass, platinum iridium, and/or any other suitable conductive material or combinations thereof. In some embodiments, the electrodes 22 may have a diameter in the range of about 5 French to about 11 French and a length of about 1 millimeter (mm) to about 4 mm, however, any suitable diameter and length may be used for electrodes, as desired. In some cases, the electrodes may be spaced apart by about 1 mm to about 10 mm, however, any suitable spacing may be used, as desired. In some embodiment, one or more conductive coils or other tissue heating device may be used in addition to or in place of ring electrodes 22.

In addition to sensing, the distal region 13 of catheter 10 can deliver ablation energy in a bipolar and/or monopolar manner. For example, radio frequency, microwave, and/or other ablative energy can be delivered via distal tip 16 from ablation source 15. In some cases, ring electrode(s) 22 and/or a separate ground pad (not shown) may act as a return electrode.

In the illustrative embodiment, handle 20 may be configured to be grasped and operated by a user. In some instances, the handle 20 may include a variety of features to facilitate control of the catheter 10 and/or mating of the catheter 10 with a fluid source 11, a control module 13, and/or an ablation source 15. In some cases, handle 20 may be configured to include at least one fitting or port 28 for mating with a source of cooling fluid 11. In some cases, the handle 20 may include a valve (not shown) for regulating the flow of fluid to the distal tip 16. In addition, the ablation catheter 10 can include an electrical connector 27 for receiving and transmitting electrical signals (e.g. ablative energy and/or control signals) to the distal tip 16 from control module 13 and/or ablation source 15. The illustrative handle 20 is merely illustrative and is not meant to be limiting in any manner. It is to be understood that any suitable handle may be used with catheter 10, as desired.

In some embodiments, handle 22 can include a control mechanism 24 for directing movement of a distal portion of elongate shaft 12. For example, catheter 10 may include a steering mechanism (shown as 44 in FIGS. 3-5) that is controlled via the proximal control mechanism 24. In one aspect, a distal section 14 of the catheter body can be deflected or bent using the steering mechanism. The steering mechanism of the elongate shaft 12 can facilitate insertion of the catheter 10 through a body lumen (e.g., vasculature) and/or placement of distal tip 16 and/or electrodes 22 at a target tissue location. In some instances, the steering mechanism can provide one or more degrees of freedom and permit up/down and/or left/right articulation. One skilled in the art will understand that the control mechanism 24 and steering mechanism of the catheter 10 can include the variety of features associated with conventional articulating catheters. For example, in some instances, a control knob 29 may be provided to control the frictional resistance for actuating, locking, and/or holding the deflection of the distal section 14.

In some examples, the ablation catheter 10 may be about 6 French to about 10 French in diameter and the portion of the catheter 10 that is inserted into the patient may be from about 60 to about 160 cm in length. In some embodiments, the length and flexibility of the catheter 10 allow the catheter to be inserted into a main vein or artery (typically the femoral vein), directed into the interior of the heart, and then manipulated such that the desired electrode(s) contact the target tissue. However, it is contemplated that any suitable diameter and length may be used for catheter 10 depending on the application. In some instances, fluoroscopic imaging may be used to provide the physician with a visual indication of the location of the catheter 10. In this instance, one or more markers (not shown) can be used, as desired.

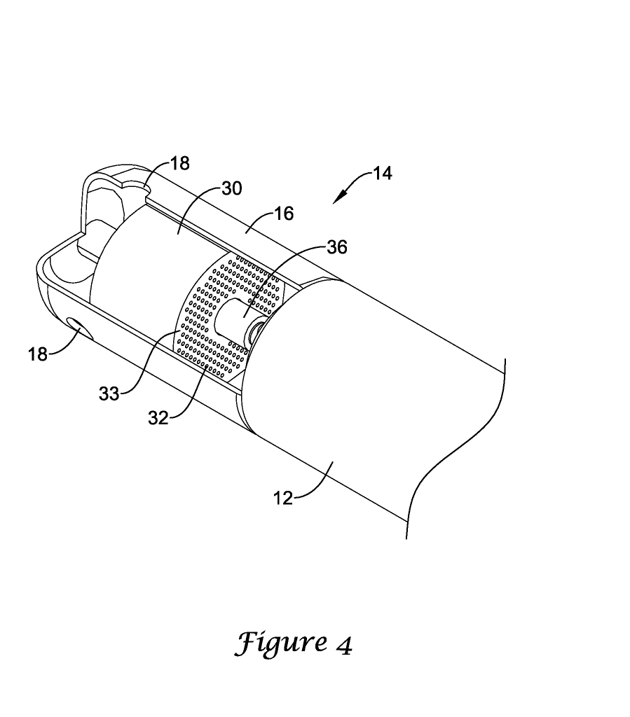

FIGS. 3 and 4 are partial cut-away views and FIG. 5 is a cross-section view of the illustrative ablation catheter 10 shown in FIG. 2. In the illustrative embodiment, the distal tip 16 may include an electrically conductive material to form, at least in part, an electrode tip of the ablation catheter 10 for delivering ablative energy to target tissue. Example electrically conductive materials can include, for example, silver, platinum, gold, stainless steel, plated brass, iridium and/or other conductive materials or combinations thereof.

As shown, the distal tip 16 may include a tubular side wall 41, a planar end wall 45, and a curved wall 43 extending between the side wall 41 to the end wall 45. In some cases, the tubular side wall 41 of distal tip 16 may include a proximal region 47 having a reduced diameter that is configured to fit into a lumen of elongated shaft 12. When so provided, an inner surface of the catheter shaft 12 can surround and mate with the outer surface of tubular side wall 41 at the area of reduced diameter (e.g. proximal region 47). However, in other examples, the distal tip 16 may be configured to form a butt joint or configured to extend over a portion of the distal section 14 of the elongated shaft 12, as desired. In any arrangement, the distal tip 16 may be secured to the distal section 14 with adhesive or other suitable instrumentality or method. For example, the distal tip 16 may be adhesively bonded, thermally bonded, soldered, or otherwise secured to the distal section 14 of elongated shaft 12.

In some embodiments, the distal tip 16 may be generally cylindrical in shape and sized for use within the heart, but this is not required. In some examples, the outer diameter of the distal tip 16 may be in the range of about 5 French to about 11 French (about 1.67 mm to about 3.67 mm) and the length of the tubular side wall 41 may be in the range of about 2 mm to about 10 mm. In some cases, a wall thickness of the distal tip 16 may be, for example, in the range of about 0.05 mm to about 0.5 mm. However, it is to be understood that the foregoing dimensions are merely illustrative and are not meant to be limiting in any manner. It is contemplated that any suitable dimensions may be used, depending on the application.

In some embodiments, a temperature sensor 36 may be mounted within the distal tip 16. In some cases, temperature sensor 36 may be a thermocouple, thermistor, or other suitable temperature sensor, as desired. As shown, temperature sensor 36 may extend proximally from the distal tip 16 and may be in electrical communication with electrical connector 27 for connection to control module 13 (shown in FIG. 2).

In the illustrative embodiment, an anchor member 42 may be mounted within the proximal region 47 of the distal tip 16. In some cases, anchor member 42 may include an electrically conductive material, such as, for example, stainless steel, or an electrically non-conductive material, such as, for example, nylon or polyimide. As shown, anchor member 42 may be generally tubular and may include a lumen. Steering mechanism 44 may be positioned within the lumen of the anchor member 42 and secured thereto along with one or more cooling lumens 38 and 40. When the anchor member 42 is electrically conductive, the portion of the steering mechanism 44 may be covered with an electrically non-conductive material, but this is not required.

In the illustrative embodiment, distal tip 16 may be electrically connected to anchor member 42 via a suitable connection, such as, for example, a solder material. As shown in FIG. 5, anchor member 42 may be electrically connected to wire 46, which may in turn be electrically connected to electrical connector 27 of the handle 20 to provide an electrical path for transmitting electrical potential to the distal tip 16. However, it is contemplated that wire 46 may be directly connected to distal tip 16 or other suitable electrical connections may be provided, if desired.

In the illustrative embodiment, ablation catheter 10 may be configured to delivery fluid to cool distal tip 16 and/or tissue that is adjacent to portions of the distal tip 16 during ablation. In some embodiment, one or more cooling tubes, such as cooling tubes 38 and 40, can be provided in the elongated shaft 12 for delivering cooling fluid to the distal tip 16. A proximal end of cooling tubes 38 and 40 may be in fluid communication with the fitting 28 for mating with the cooling fluid source 11. In some embodiments, handle 20 may include a valve (not shown) for regulating the flow of cooling fluid through cooling tubes 38 and 40 to the distal tip. In some embodiments, cooling tubes 38 and 40 may be secured relative to distal tip 16 using anchor member 42. When anchor member 42 is electrically conductive, an insulating layer (not shown) can be provided, if desired. However, other manners of securing cooling tubes 38 and 40 in elongate shaft 12 may be used. Further, it is contemplated that one, two, three, four, or any other number of cooling tubes may be provided.

In the illustrative embodiment, distal tip 16 may include one or more cooling chamber into which the cooling fluid is delivered, such as, for example, proximal cooling chamber 60 and distal cooling chamber 62. In some embodiments, proximal cooling chamber 60 and distal cooling chamber 62 can be separated by filter or porous member 30. However, in other embodiments, a thermal mass or other suitable structure may be used to separate the proximal and distal cooling chambers 60 and 62. The cooling chambers 60 and 62 may be configured for cooling hotspots associated with conventional ablation catheters. For example, the cooling chambers 60 and 62 can receive a flow of fluid to draw heat away from the side wall 41 and end wall 45 of the distal tip 16, such as, for example, a portion of the distal tip 16 adjacent to the catheter shaft 12 where RF current may tend to concentrate. Cooling fluid may be configured to enter proximal cooling chamber 60 via cooling tubes 38 and/or 40 and then flow into distal cooling chamber 62 via a plurality of pores 32 in porous member 30. Cooling fluid may exit the catheter 10 through the one or more fluid outlets, or irrigation apertures 18, positioned in the tubular side wall 41 and/or end wall 45 of the distal tip 16. For example, the distal tip 16 may include six irrigation apertures, however, any suitable number of irrigation apertures 18 may be used, such as 1, 2, 3, 4, 5, 6, 7, 8, 9, 10, or any other number of irrigation apertures 18, as desired. In such an arrangement, the ablation catheter 10 can be considered as having an open-loop configuration in which cooling fluid exits the device through tip 16.

As shown in FIGS. 3-5, the irrigation apertures 18 may be formed in a direction substantially orthogonal to a longitudinal axis of the elongate shaft 12 to promote circulation and/or swirling of the fluid around an exterior of the distal tip 16 to help reduce coagulation formation and/or to help reduce blood concentration adjacent to tip 16. However, in another instance, the irrigation apertures 18 may be positioned parallel to the longitudinal axis of the catheter shaft 12, or a combination of parallel and orthogonal to the longitudinal axis, as desired.

In the illustrative embodiment, the cooling fluid may be configured to cool the distal tip 16 and/or the tissue adjacent to the distal tip 16. In some cases, cooling fluid may circulate within the proximal cooling chamber 60 and within distal cooling chamber 62 to aid the cooling.

In some embodiments, decreasing the temperature of the distal tip 16 and/or adjacent tissue with cooling fluid may help reduce the likelihood that the tissue in contact with the distal tip 16 will char and/or that coagulum will form on the surface of the distal tip 16. As such, the amount of energy supplied to the tissue may be increased, and the energy may be transferred to the tissue more efficiently, as compared to an ablation catheter that does not include fluid cooling. This may result in the formation of larger and deeper lesions. In addition to cooling tissue adjacent to the distal tip 16, fluid that exits the distal tip 16 may also sweep biological material, such as blood and tissue away from the distal tip 16, further reducing the likelihood of coagulum formation, which can result in less effective energy transfer to the tissue.

As shown in FIG. 3-5, porous member 30 may include a proximal end 33, a distal end 31, and a plurality of pores 32 extending therethrough. Porous member 30 may be configured to filter, break-up, reduce, and/or remove bubbles in the cooling fluid prior to the fluid exiting the ablation catheter through irrigation apertures 18. In some cases, all or substantially all of the fluid that may exit through irrigation apertures 18 may be filtered through porous member 30. As shown, porous member 30 may be sized and/or configured to substantially fill the cross-sectional area of the distal tip 16. In some cases, porous member 30 may also be fluidly or substantially fluidly sealed to the side wall 41 and/or temperature sensor 36. In any event, porous member 30 may be configured such that bubbles that may potentially pose a risk of causing air embolisms in a vessel or other portion of the body may not exit irrigation apertures 18.

In some embodiments, at least some or all of the plurality of pores 32 may be oriented in a generally parallel configuration and/or have a generally uniform diameter. However, in other embodiments, at least some or all of the plurality of pores 32 may be oriented in a generally non-parallel configuration and/or have a generally non-uniform diameter. The plurality of pores 32 may be sized to have a diameter that is capable of filtering out bubbles. For example, the diameter of the plurality of pores 32 may be on the order of micrometers, which may be referred to as micro-pores. However, it is contemplated that any suitable diameter may be used that may break-up bubbles such that any remaining bubbles may not pose a significant risk of causing air embolisms in the vessel or other portion of the body.

In some embodiments, the porous member 30 may include any suitable porous material that can break-up and/or reduce bubbles while allowing a fluid flow therethrough. Example material may include, but are not limited to, a fabric, a membrane, a woven mesh, a non-woven fiber, a sintered material, a porous fiber such as a porous carbon fiber, and/or any other suitable porous material, as desired. The porous member 30 may include, for example, a metal, a ceramic, and/or a polymer. Example porous polymer materials may include, for example, thermoset polymers, thermoplastic polymers, elastomer materials, organic or synthetic materials, and any other suitable polymer material, as desired. However, the foregoing materials are merely illustrative and are not meant to be limiting in any manner. It is to be understood that any suitable porous material may be used for porous member 30, as desired.

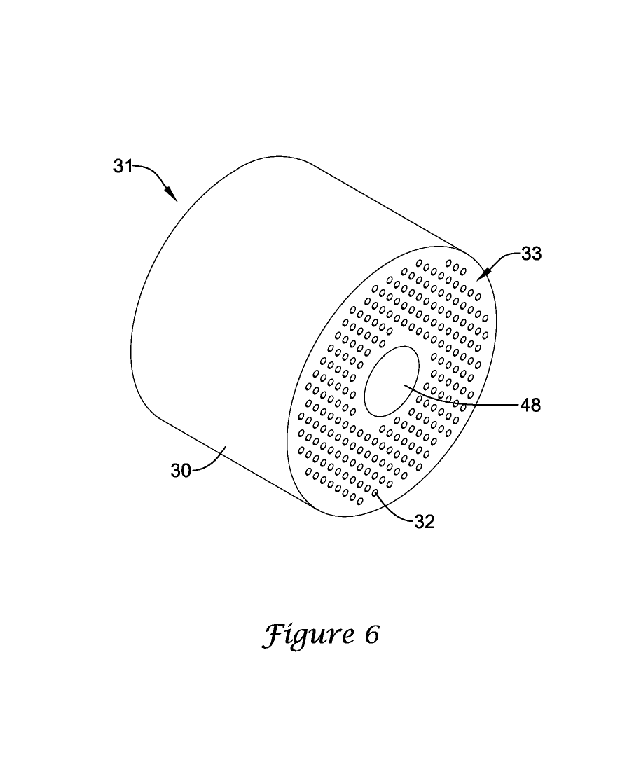

FIG. 6 is a perspective view of an illustrative porous member 30. In some cases, porous member 30 may be used in conjunction with the ablation catheter shown in FIGS. 2-5. As shown, porous member 30 may include a plurality of pores 32 extending between ends 31 and 33. Porous member 30 may also include a central opening 48 configured to receive temperature sensor 36 therein. In some cases, central opening 48 may be configured to fluidly seal to temperature sensor 36, but this is not required. The plurality of pores 32 may be sized to allow fluid to flow therethrough and may be configured to break-up and/or reduce bubbles in the fluid to help reduce the risk of forming air embolisms in the vessel or body.

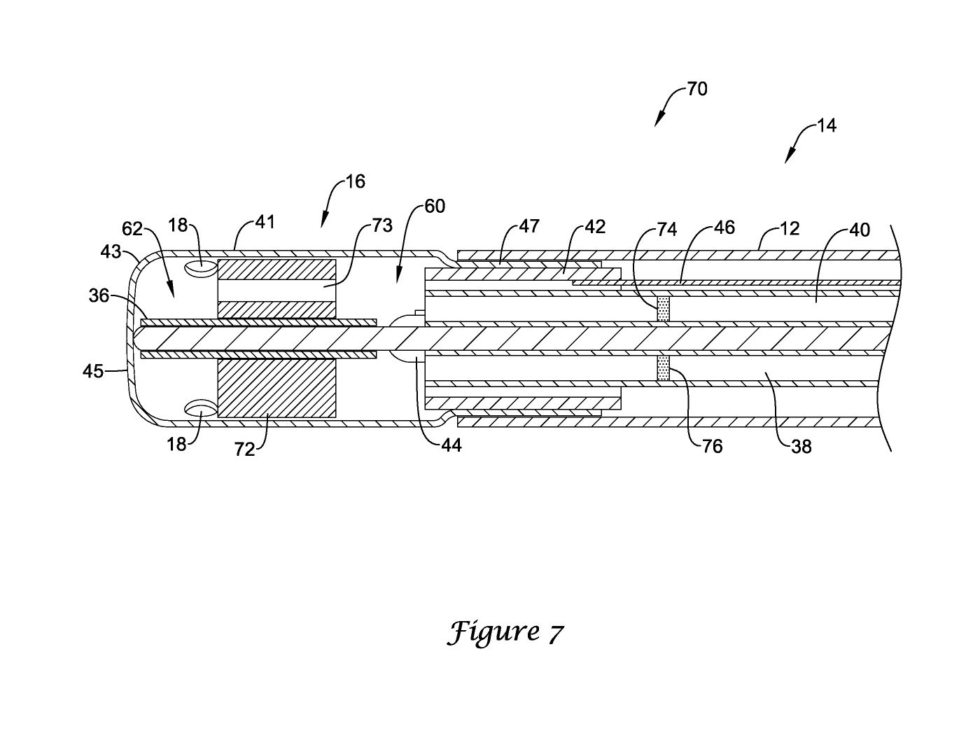

FIG. 7 is a cross-sectional view of another illustrative ablation catheter 70. The ablation catheter 70 may be similar to the ablation catheter 10 except that porous member 30 is replaced with thermal mass 72 and porous members 74 and 76 are provided in a portion of cooling lumens 38 and 40. As illustrated, the thermal mass 72 may separate the distal tip 16 into proximal chamber 60 and distal chamber 62. In some cases, thermal mass 72 may be electrically conductive and/or thermally conductive. Example electrically and thermally conductive materials may include, for example, brass, copper, stainless steel, and combinations thereof. However, other materials may be used, as desired. In some cases, thermal mass may be thermally conductive, but not necessarily electrically conductive, if desired. Thermal mass 60 may include a fluid passageway to permit fluid to flow from the proximal chamber 60 to distal chamber 62.

Porous members 74 and 76 may include any suitable porous material that can break-up and/or reduce bubbles while allowing a fluid flow therethrough. Example materials may include, but are not limited to, a fabric, a membrane, a woven mesh, a non-woven fiber, a sintered material, a porous fiber such as a porous carbon fiber, and/or any other suitable porous material, as desired. The porous members 74 and 76 may include, for example, a metal, a ceramic, and/or a polymer. Example porous polymer materials may include, for example, thermoset polymers, thermoplastic polymers, elastomer materials, organic or synthetic materials, and any other suitable polymer material, as desired. However, the foregoing materials are merely illustrative and are not meant to be limiting in any manner. It is to be understood that any suitable porous material may be used for filters 74 and 76, as desired.

Further, it is contemplated that porous members 74 and 76 may be positioned at a distal end of cooling tubes 38 and 40, in a proximal region of the cooling tubes 38 and 40, or at any other location in the ablation catheter 70, as desired.

While the foregoing has been described with reference to ablation catheters, this is not meant to be limiting in any manner. It is contemplated that the filter may be provided in any suitable fluid delivery device to reduce the flow of bubbles into a blood vessel. In some cased, the filter may be included in any fluid delivery device that poses a risk of causing air embolisms.

Having thus described the preferred embodiments of the present disclosure, those of skill in the art will readily appreciate that yet other embodiments may be made and used within the scope of the claims hereto attached. Numerous advantages of the disclosure covered by this document have been set forth in the foregoing description. It will be understood, however, that this disclosure is, in many respect, only illustrative. Changes may be made in details, particularly in matters of shape, size, and arrangement of parts without exceeding the scope of the disclosure. The invention's scope is, of course, defined in the language in which the appended claims are expressed.

What is claimed is:

1. A medical device comprising:
an elongated shaft including a proximal region and a distal region, wherein the proximal region of the elongated shaft is configured to be coupled to a fluid source for receiving a cooling fluid, wherein the elongated shaft defines at least one cooling lumen in fluid communication with the fluid source for supplying the cooling fluid to the distal region of the catheter shaft;
an electrode tip positioned adjacent to the distal region of the elongated shaft, the electrode tip including a wall defining a cooling chamber that is in fluid communication with the at least one cooling lumen, the wall including one or more irrigation apertures for expelling the cooling fluid from the cooling chamber of the electrode tip; and
a porous member disposed in the cooling chamber or the at least one cooling lumen such that substantially all of the cooling fluid that is expelled through the one or more irrigation apertures flows through the porous member prior to being expelled, wherein the porous member includes a plurality of pores sized and configured to filter, reduce, and/or break-up bubble formations in the cooling fluid such that bubble formations posing a risk of forming embolisms in a vessel or body cavity are not expelled through the one or more irrigation apertures into the vessel or body cavity.

2. The medical device of claim 1, wherein the plurality of pores are a plurality of micro-pores having diameters on the order of micrometers.

3. The medical device of claim 1, wherein the porous member is positioned in the cooling chamber and is configured to divide the cooling chamber into a proximal chamber and a distal chamber, wherein the proximal chamber is in fluid communication with the at least one cooling lumen and the distal chamber is in fluid communication with the proximal chamber via the plurality of pores.

4. The medical device of claim 3, wherein the one or more irrigation apertures are formed in the wall of the electrode tip in the distal chamber.

5. The medical device of claim 3, wherein the proximal chamber is configured to promote circulation of the cooling fluid.

6. The medical device of claim 1, wherein the porous member is positioned in the at least one cooling lumen.

7. The medical device of claim 6, wherein the porous member is positioned proximal of the electrode tip.

8. The medical device of claim 1, wherein the electrode tip is in electrical communication with the proximal region of the elongated shaft via one or more conductive members extending through the elongated shaft.

9. The medical device of claim 8, further comprising one or more ring electrodes disposed around the distal region of the elongated shaft proximal of the electrode tip.

10. The medical device of claim 1, wherein the plurality of pores of the porous member extend from a proximal end of the porous member to a distal end of the porous member.

11. The medical device of claim 10, wherein the plurality of pores are configured in a generally parallel orientation.

12. The medical device of claim 10, wherein each of the plurality of pores are configured to have substantially the same diameter.

13. The medical device of claim 1, wherein the porous member includes woven or non-woven fibers.

14. The medical device of claim 1, wherein the porous member includes a sintered material.

15. A medical device comprising:
a catheter including a proximal region and a distal region, the proximal region of the catheter including a coupling configured to couple to a fluid source for receiving a fluid, the distal region of the catheter including one or more irrigation apertures for expelling the fluid from the catheter, the catheter defining a fluid path extending between the coupling and the one or more irrigation apertures; and
a porous member including a plurality of micro-pores, the porous member positioned at a location in the fluid path and configured to substantially fill the cross-sectional area of the location in the fluid path such that the fluid being expelled from the catheter via the one or more irrigation apertures flows through the plurality of micro-pores.

16. The medical device of claim 15, wherein the catheter includes an electrode distal tip for ablating tissue, wherein location in the fluid path of the porous member is in the electrode distal tip.

17. The medical device of claim 15, wherein the catheter includes an electrode distal tip for ablating tissue, wherein location in the fluid path of the porous member is proximal of the electrode distal tip.

18. A method of delivering a fluid to a treatment site with a fluid delivery device, the method comprising:
coupling a proximal end of the fluid delivery device to a fluid source;
providing a fluid flow through a fluid path of the fluid delivery device;
passing the fluid flow through a porous member in the fluid path to filter the fluid flow for bubble formations; and
expelling the filtered fluid flow from the fluid delivery device through one or more irrigation apertures.

19. The method of claim 18, wherein the porous member includes a plurality of pores having diameters on the order of micrometers.

20. The method of claim 18, further comprising:
providing an electrical signal to an electrode positioned in the distal region of the fluid delivery device via one or more electrical conductors; and
ablating tissue adjacent to the distal region of the fluid delivery device.
